(19) United States
(12) Patent Application Publication
(10) Pub. No.: US 2014/0128415 A1
(43) Pub. Date: May 8, 2014

(54) EXCIPIENT DRUG COMPOSITION

(71) Applicant: Paul Daniel Yered, Thousand Oaks, CA (US)

(72) Inventor: Paul Daniel Yered, Thousand Oaks, CA (US)

(21) Appl. No.: 14/150,685

(22) Filed: Jan. 8, 2014

Related U.S. Application Data
(63) Continuation-in-part of application No. 13/484,046, filed on May 30, 2012.

(51) Int. Cl. A61K 47/38; A61K 47/22; A61K 47/36

(52) U.S. Cl. CPC A61K 47/38; A61K 47/36; A61K 47/22
USPC 514/263.34; 514/570; 514/630; 514/653; 514/548

(57) ABSTRACT

A excipient drug composition is disclosed. The excipient drug composition may include a vegetable cellulose or a fruit cellulose, an active ingredient and a genetically modified organism free starch. The vegetable cellulose or fruit cellulose may be derived from wood free vegetable pulp and the fruit cellulose is a wood pulp free cellulose. The genetically modified organism free starch may be selected from the group consisting of a genetically modified organism free rice starch, a genetically modified organism free potato starch or a genetically modified organism free pea starch.

This application claims priority to U.S. Provisional Application 61/311,324 filed on Mar. 6, 2010, the entire disclosure of which is incorporated by reference. The present application is a continuation-in-part of U.S. Non-Provisional Patent Application No. 13/484,046 filed on May 30, 2012, the entire disclosure of which is incorporated by reference.

### TECHNICAL FIELD & BACKGROUND

Most prescription drugs and many over the counter medications contain excipients that may also be referred to as inactive ingredients, such as polysaccharides and other chemically processed artificial additives. Many of these inactive ingredients are commonly added to foods by the food industry. For example, many foods contain artificial flavors, dyes, and other additives such as xanthan gum and various sugars, which are also found in medications. It is believed but often questionable that an excipient or an inactive ingredient is sometimes needed to deliver a drug or an active ingredient to the site of drug interaction when taken orally to facilitate what is referred to as absorption, distribution, metabolism and excretion or ADME while intravenous drugs do not require these inactive ingredients and therefore show relatively better and quicker results. However, most drugs undergo ADME without the need for excipients. The excipients are mainly utilized as fillers or bulking agents, to increase oral dosage accuracy in terms of milligrams or micrograms measurements or to induce specific colors to the tablets. The ratio of a drug and it's excipients in addition to any food additives ingested may be important with regard to the efficacy of a drug, considering many excipients are the same as or directly or indirectly related to many food additives.

These drug additives may result in unwanted impurities and consequences. Metformin, for example, is meant to lower a diabetic's blood sugar, but at the same time, contains polysaccharides which may have the opposite effect. Warfarin may be meant to thin the blood but contains modified cornstarch, which also has the opposite effect. Furthermore the inactive ingredients may form unwanted complexes with or without artificial food additives after continued administration, which may result in autoimmune conditions. The processing of excipients may contribute impurities and possibly toxins to the medications. For example, many starches and polysaccharides are washed with chlorine or hypochlorite, dextrins, phosphates and other synthetic acids and organisms.

The medical field continues to recognize the need for improving results from drug therapies, in addition to improving the efficacy of each drug. The obvious advantage may be to improve compliance through including less allergens or allergen precursors and/or decrease the amount and time at which the patient must be prescribed the drug to minimize any side effects and/or adverse effects that may occur. The medications may be made potentially more tolerable, effective and healthier by including sources of cellulose that are not from pine trees and wood pulp based with limited and more natural processing aids and reagents. More importantly the patient may be exposed to less chemically treated and modified polysaccharides and more nutrients that have not been modified.

Many foods contain chemicals and/or toxins such as food additives. It may be well known that many of these food chemicals have been shown to be carcinogenic (i.e. Saccharin and yellow dye 6). Many of these are food additives, artificial additives, artificial flavors, artificial colors, natural flavors and/or inactive ingredients, many of which are found in tablets, capsules, liquid medications, candies and drinks. Starches, one of the most common drug and food additives are often genetically engineered and chemically treated with reagents and processing aids, leaving many residues and/or composites on the final product. Many products also list natural flavors when they also contain chemicals and may be prepared and divided into sub-types based on the amount and type of chemicals utilized in each preparation. Therefore their purity level varies.

Starches are often genetically engineered and chemically treated. Once ingested, their parent compound and/or by-product incubation and/or fermentation may occur, completing and/or beginning another possible food additive-inactive ingredient complex continued cycle.

Some food and drug additives may result in substrates for drug therapies, since many of the prescribed drugs contain chemical side groups that may correlate with the chemistry of these additives. Many of the drug rings and side groups correlate with the chemical structure of food additives and inactive ingredient food additive complexes should be considered.

The medical field continues to recognize the need for improving results from drug therapies, in addition to improving the efficacy of each drug therapy. The advantage may be to improve compliance and/or decrease the amount and time at which the patient must be prescribed the drug to minimize any adverse and/or side effects that may occur.

Since many foods contain similar additives as those found in medications a decrease in the amount of commonly utilized inactive ingredients has shown more compliance with fewer side effects in certain patients.

Several drug formulations have been created in an attempt to increase the delivery and efficacy of drugs. The need for continued manufacturing of new drugs may result from the continued artificial food consumption and drug re-administration of inactive ingredients to patients. By including less modified polysaccharide starches and non-genetically and chemically treated additives, the patient may be less likely to show adverse and side effects and improve compliance.

The present invention generally relates to a drug formulation. More specifically, the invention includes an excipient drug composition from a combination or real edible vegetable and/or fruit wherein a pine tree is considered a real edible vegetable.

It is an object of the present invention to provide an excipient drug composition for all active ingredients that includes non-genetically modified starches, non-wood pulp based cellulose, GMO free whole food powders and non-hydrogenated oils, whether alone or in combination.

It is an object of the present invention to provide an excipient drug composition for drug therapy utilizing minimally processed powders and oils, as opposed to utilizing chemically treated polysaccharides and/or complex modified artificial sugars. The treated powders that may be genetically and/or chemically treated are oxidized. Chemically modified and/or treated polysaccharides may form unwanted complexes with food additives such as monosodium glutamate and/or other related food chemicals as seen in their chemical side groups.

Many patients being treated with current formulations may experience a rebound effect such as rebound headaches, when in fact many are allergic to the modified cornstarch, along with other wood cellulose and inactive ingredients that may cause unwanted complexes and cause an allergic or autoimmune response. The question remains whether this rebound effect may also relate to other organ responses and conditions.

It is an object of the present invention to provide an excipient drug composition that reduces adverse and side effects and increases the efficacy from other drugs currently taken by a user.

What is really needed is an excipient drug composition that provides consumers whom are sensitive to current additives, wood pulp products, gluten and other food sensitivities an alternative therapy.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

The excipient drug composition may be a hypoallergenic drug formulation may be to provide inpatient and outpatient drug therapy utilizing the above mentioned criteria (i.e. non-wood based cellulose and GMO free starches) from a real edible vegetable source other than wood pulp or in addition to the commonly utilized potato as a source or a combination of these ingredients. The hypoallergenic drug formulation may be utilized with a variety of drugs such as those for sinus, allergy, asthma, migraine headache, anti-coagulant (i.e., warfarin) and oral antidiabetic drugs (i.e., metformin).

With hypoallergenic drug formulations the continued utilization of genetically and chemically treated starches, wood pulp based cellulose and proteins may be minimized, avoided and replaced with organic oils cold or expeller pressed oils, alternative real vegetable starches, such as organically processed rice starch along with other sources of vegetable starches, non-wood based cellulose from sources of a variety of natural edible fruits and vegetables, excluding the utilization of phosphates, dextrins, absolute solvent extractions and synthetic acids. Antipsychotic, antidepressant and antiepileptic drugs may also be utilized with the hypoallergenic drug formulations, along with many other drugs.

### Examples of Some Modified or Artificial Food Additives

Most modified or artificial food additives have been genetically modified or GMO and later chemically washed with artificial synthetic acids and other chemicals such as dextrin like precursors, phosphates and/or chlorine such as hypochlorite as opposed to lime, ethanol and other real nutrients.
Examples include:
Glucose in ring form and chain form
Sucrose, Dextrin or Dextrose
Modified Food Starch (i.e., D-Glucose units)
Malt Dextrin from Corn
Corn Syrup
High Fructose Corn Syrup
Corn Starch
Pre-gelatinized Corn Starch
Sodium Starch Glycolate
Monosodium Glutamate
Sodium Nitrite
Erythrobate
Dyes (i.e., Red-Yellow and others)
Xanthan Gum
Sodium Benzoate
Saccharin
Phenylalanine
All artificial Proteins and Amino Acids
Aspartame
Guanylate Salts
Modified chemically treated oils (i.e., solvent extractions and hydrogenated oils)

### Examples of Some GMO and/or Chemically Treated Inactive Ingredients

Cross-utilization accumulation may occur with food additive and drug excipients such as xanthan gum, color dyes, corn syrups and starches. Other examples include:
Corn Starch
Pre-gelatinized Corn Starch
Hydroxymethylcellulose
Methyl Cellulose
Sodium Benzoate
Xanthan Gum
Lactose Monohydrate
Polyethylene Glycol
Microcrystalline Cellulose
Povidone
FDC Colors Aluminum Lakes
Silicon Dioxide
Sodium Lauryl Sulfate
Titanium Dioxide

### Examples of Sources of Cellulose and Starches or Whole Powders Other Than Wood Pulp Based

Sources of cellulose and starches or whole powders other than wood pulp include carrot, cilantro, rice, potato, corn, pea such as green pea and chick pea, celery, Brussels sprouts, blueberry, tapioca, bean (i.e., favas, lentils, lima, garbanzo, navy, mung, soy and other suitable beans), banana, arrowroot (i.e., Polynesian), arracacha, barley, oat, millet, rye, breadfruit, buckwheat, canna, colacasia, katakuri, kudzu, malanga, orca, sago, sorghum, sweet potato, taro, chestnut, water chestnut, yams, beet, cassava, cauliflower, parsnip, plantain, rutabaga, pumpkin, squash, winter squash and turnip and fig.

### Examples of Expeller Pressed or Cold Pressed Oils

Expeller pressed or cold pressed oils may include almond, sesame, avocado, coconut, olive and other suitable expeller pressed or cold pressed oils.

The excipient drug composition may include a vegetable cellulose or a fruit cellulose and an active ingredient. The vegetable cellulose may be derived from wood free vegetable pulp. The fruit cellulose may be a wood pulp free cellulose. The excipient drug composition may also include a genetically modified organism free starch. The excipient drug composition may include a vitamin E preservative or a vitamin C preservative. The active ingredient may be ibuprofen. The active ingredient may be acetaminophen. The active ingredient may be selected from the group consisting of acetaminophen and ibuprofen or other suitable active ingredient. The active ingredient may be selected from the group consisting of acetaminophen, ibuprofen and phenylephrine. The active ingredient may be selected from the group consisting of acetaminophen, ibuprofen and pseudoephedrine. The active ingredient may be selected from the group consisting of acetaminophen, aspirin and caffeine. The excipient drug composition may be contained in a capsule or a tablet.

An excipient drug composition may be consisting of a vegetable cellulose or a fruit cellulose, an active ingredient and a genetically modified organism free starch. The vegetable cellulose may be derived from wood free vegetable pulp. The fruit cellulose may be a wood pulp free cellulose. The active ingredient may be ibuprofen in the approximate range of 40 mg to 800 mg. The active ingredient may be acetaminophen in the approximate range of 40 mg to 1000 mg. The active ingredient may be acetaminophen in the approximate range of 40 mg to 1000 mg and ibuprofen in the approximate range of 40 mg to 800 mg. The active ingredient may be acetaminophen in the range of approximate 40 mg to 1000 mg, ibuprofen in the approximate range of 40 mg to 800 mg and phenylephrine in the approximate range of range 5 mg to 20 mg. The active ingredient may be acetaminophen in the approximate range of 40 mg to 1000 mg, ibuprofen in the approximate range of 40 mg to 800 mg and pseudoephedrine in the approximate range of 10 mg to 120 mg. The genetically modified organism free starch may be selected from the group consisting of a genetically modified organism free rice starch, a genetically modified organism free potato starch or a genetically modified organism free pea starch. The excipient drug composition may also include a vitamin E preservative or a vitamin C preservative. The vitamin E preservative or the vitamin C preservative may be an oil. The vitamin E preservative or the vitamin C preservative may be a preservative powder. The excipient drug composition may be contained in a capsule or a tablet.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. An excipient drug composition, comprising:
a vegetable cellulose or a fruit cellulose; and
an active ingredient.

2. The excipient drug composition according to claim 1, wherein the vegetable cellulose is derived from wood free vegetable pulp.

3. The excipient drug composition according to claim 1, wherein the fruit cellulose is a wood pulp free cellulose.

4. The excipient drug composition according to claim 1, further comprising a genetically modified organism free starch.

5. The excipient drug composition according to claim 1, further comprising a vitamin E preservative or a vitamin C preservative.

6. The excipient drug composition according to claim 1, wherein the active ingredient is ibuprofen.

7. The excipient drug composition according to claim 1, wherein the active ingredient is acetaminophen.

8. The excipient drug composition according to claim 1, wherein the active ingredient is selected from the group consisting of acetaminophen and ibuprofen.

9. The excipient drug composition according to claim 1, wherein the active ingredient is selected from the group consisting of acetaminophen, ibuprofen and phenylephrine.

10. The excipient drug composition according to claim 1, wherein the active ingredient is selected from the group consisting of acetaminophen, ibuprofen and pseudoephedrine.

11. The excipient drug composition according to claim 1, wherein the active ingredient is selected from the group consisting of acetaminophen, aspirin and caffeine.

12. The excipient drug composition according to claim 1, wherein the excipient drug composition is contained in a capsule or a tablet.

13. An excipient drug composition, consisting of:
a vegetable cellulose or a fruit cellulose;
an active ingredient; and
a genetically modified organism free starch.

14. The excipient drug composition according to claim 13, wherein the vegetable cellulose is derived from wood free vegetable pulp.

15. The excipient drug composition according to claim 13, wherein the fruit cellulose is a wood pulp free cellulose.

16. The excipient drug composition according to claim 13, wherein the active ingredient is ibuprofen in the range of 40 mg to 800 mg.

17. The excipient drug composition according to claim 13, wherein the active ingredient is acetaminophen in the range of 40 mg to 1000 mg.

18. The excipient drug composition according to claim 13, wherein the active ingredient is acetaminophen in the range of 40 mg to 1000 mg and ibuprofen in the range of 40 mg to 800 mg.

19. The excipient drug composition according to claim 13, wherein the active ingredient is acetaminophen in the range of 40 mg to 1000 mg, ibuprofen in the range of 40 mg to 800 mg and phenylephrine in the range of range 5 mg to 20 mg.

20. The excipient drug composition according to claim 13, wherein the active ingredient is acetaminophen in the range of 40 mg to 1000 mg, ibuprofen in the range of 40 mg to 800 mg and pseudoephedrine in the range of 10 mg to 120 mg.

21. The excipient drug composition according to claim 13, wherein the genetically modified organism free starch is selected from the group consisting of a genetically modified organism free rice starch, a genetically modified organism free potato starch or a genetically modified organism free pea starch.

22. The excipient drug composition according to claim 13, further comprising a vitamin E preservative or a vitamin C preservative.

23. The excipient drug composition according to claim 22, wherein the vitamin E preservative or the vitamin C preservative is an oil.

24. The excipient drug composition according to claim 22, wherein the vitamin E preservative or the vitamin C preservative is a preservative powder.

25. The excipient drug composition according to claim 13, wherein the excipient drug composition is contained in a capsule or a tablet.
